(19) United States
(12) Patent Application Publication
(10) Pub. No.: US 2016/0286790 A1
(43) Pub. Date: Oct. 6, 2016

(54) APPARATUS FOR OBSERVING CORNEA FOR TRANSPLANTATION AND OBSERVATION SYSTEM HAVING THE SAME

(71) Applicant: KONAN MEDICAL, INC., Nishinomiya-shi (JP)

(72) Inventors: Hidekazu Yamamoto, Nishinomiya-shi (JP); Koichi Kondo, Nishinomiya-shi (JP)

(21) Appl. No.: 15/085,227

(22) Filed: Mar. 30, 2016

(30) Foreign Application Priority Data
Mar. 31, 2015 (JP) 2015-071956

(51) Int. Cl. A01N 1/02; G02B 21/00; C12N 5/079; G02B 7/02; A61F 2/00; G02B 21/36; G02B 21/02

(52) U.S. Cl. CPC A01N 1/0263; G02B 21/36; G02B 21/0012; G02B 21/02; G02B 7/023; A61F 2/0095; C12N 5/0621

(57) ABSTRACT

A finder function of an imaging unit for taking an entire image including a cornea for transplantation displays a high-quality image. There are provided a support table for supporting an observation container containing a cornea for transplantation as an observed target, a specular optical system that includes a first light source and a first imaging unit, and includes an illuminating optical system and an imaging optical system for acquiring and observing a magnified image of a cornea for transplantation from above or below the observation container in the vertical direction by specular reflection, a second imaging unit for taking an entire image including a region in which the cornea for transplantation is contained from above or below the observation container in the vertical direction, and a switch for selectively switching between the first imaging unit and the second imaging unit to acquire the magnified image and the entire image.

### CROSS-REFERENCE TO RELATED APPLICATION

This application claims priority to Japanese Patent Application No. 2015-071956 filed on Mar. 31, 2015, the disclosure of which including the specification, the drawings, and the claims is hereby incorporated by reference in its entirety.

### BACKGROUND OF THE INVENTION

1. Field of the Invention

The present invention relates to an apparatus for observing cornea for transplantation and an observation system, the apparatus including:

a support table configured to support an observation container containing a cornea for transplantation as an observed target;

a specular optical system including an illuminating optical system and an imaging optical system, the specular optical system having a first light source and a first imaging unit, and being configured to acquire and observe a magnified image of the cornea for transplantation from above or below the observation container in the vertical direction by specular reflection;

a second imaging unit configured to an entire image from above or below the observation container in the vertical direction, the entire image including a region where the cornea for transplantation is contained.

2. Description of the Related Art

A system for observing cornea for transplantation disclosed in a below-mentioned patent document is known as an observation apparatus and an observation system. This system includes a first light source for illuminating a cornea for transplantation contained in an observation container, and an entire-view imaging means (camera) for imaging an entire image including an illuminated region.

With this configuration, the position of the cornea for transplantation can be readily checked while observing a magnified image. Since the magnified image and the entire image are simultaneously displaced on a monitor screen, an observed region of the cornea for transplantation can be visually recognized with ease.

### PRIOR ART DOCUMENT

### Patent Document

Patent document 1: JP-A-2011-115556

### SUMMARY OF THE INVENTION

However, the above conventional art has a following problem. A specular optical system is disposed below the observation container in the vertical direction. However, the camera takes the entire image from obliquely below the observation container. In this case, when the observation container is horizontally (forward and rearward or right and left) moved to change the observed position and then, is vertically (upward and downward) moved for focusing, the position of the entire image is disadvantageously displaced. Thus, the position display function of a finder of the camera is unsatisfactory. Further, oblique observing may lead to a deformed or out-of-focus image.

### In

In consideration of such situation, an object of the present invention is provide an apparatus for observing cornea for transplantation and an observation system that can improve a finder function of an imaging unit for imaging an entire image including a cornea for transplantation, and display a high-quality image.

To attain the object, an apparatus for observing cornea for transplantation according to the present invention includes:

a support table configured to support an observation container containing a cornea for transplantation as an observed target;

a specular optical system including an illuminating optical system and an imaging optical system, the specular optical system having a first light source and a first imaging unit, and being configured to acquire and observe a magnified image of the cornea for transplantation from above or below the observation container in the vertical direction by specular reflection;

a second imaging unit configured to an entire image from above or below the observation container in the vertical direction, the entire image including a region where the cornea for transplantation is contained; and

a switch configured to selectively switch between the first imaging unit and the second imaging unit to acquire the magnified image or the entire image.

Actions and effects of the apparatus for observing cornea for transplantation thus configured will be described below. The magnified image of the cornea for transplantation contained in the observation container is taken by the first imaging unit of the specular optical system. The entire image of the cornea for transplantation is taken by the second imaging unit. The operator can select which image to acquire using the first imaging unit or the second imaging unit, and the switch for switching to the selected imaging unit is provided. When the first imaging unit is selected, the magnified image can be taken from above or below the observation container in the vertical direction, and when the second imaging unit is selected, the entire image can be taken from above or below the observation container in the vertical direction. Therefore, problems caused by taking the entire image obliquely, that is, displacement and deformation of the entire image can be solved. As a result, a finder function of the second imaging unit for taking the entire image can be improved to display a high-quality image.

Preferably, the switching mechanism of the switch of the present invention has a mechanism configured to integrally move the second imaging unit and at least a part of the specular optical system.

The switching mechanism can be configured in various manners. By integrally moving at second imaging unit and at least a part of the specular optical system, the second imaging unit can be moved to a space in which at least a part of the specular optical system is disposed to locate the second imaging unit above or below the observation container in the vertical direction.

Preferably, the switching mechanism of the present invention has a mechanism configured to integrally move an objective lens system constituting the specular optical system and the second imaging unit.

With this configuration, the second imaging unit advances and retracts in an optical path of the specular optical system, suppressing the dimension of the unit to be moved.

Preferably, the switching mechanism of the present invention has a mechanism configured to integrally move the entire specular optical system and the second imaging unit.

When moving the specular optical system, by moving the entire specular optical system, the second imaging unit can be located above or below the observation container in the vertical direction.

Preferably, the switching mechanism of the present invention has a mechanism configured to advance and retract the second imaging unit in an optical path of the specular optical system.

With this configuration, since only the second imaging unit is moved, configuration of the switching mechanism can be simplified.

Preferably, the switching mechanism of the present invention has a mechanism configured to move the observation container.

Switching can be made by moving the observation container rather than by moving the second imaging unit and the specular optical system. Since it is no need to move the specular optical system, upsizing of the switching mechanism can be prevented.

Preferably, a switching detection means configured to detect switching of the imaging unit by the switching mechanism is provided. With this configuration, it is possible to recognize which of the imaging units is selected, controlling display on the monitor.

According to the present invention, preferably, a second light source configured to illuminate the cornea for transplantation from above or below the observation container; and an observation-container moving mechanism configured to move the observation container in a thickness direction of the cornea for transplantation are provided, and an endothelium, an epithelium, a portion between the endothelium and the epithelium of the cornea for transplantation can be observed.

By providing the observation-container moving mechanism, the specular optical system can focus on the endothelium and the epithelium to observe the endothelium, the epithelium, the portion between the endothelium and the epithelium. Since the illuminating optical system of the specular optical system may not observe the epithelium, by providing the second light source, the endothelium as well as the epithelium, and the portion between the endothelium and the epithelium can be readily observed.

A system for observing cornea for transplantation according to the present invention includes an apparatus for observing cornea for transplantation according to the present invention, and a control unit configured to control operation of each component of the observation apparatus, and the control unit has alight source control configured to turn on or enhance the second light source when a predetermined amount of movement of the observation container by the observation-container moving mechanism to observe an epithelium is detected.

To shift observation of the endothelium to observation of the epithelium, the observation-container moving mechanism needs to move the observation container. To observe the epithelium, as described above, it is needed to use the second light source. A distance between the endothelium and the epithelium can be previously inputted in consideration of the typical thickness of the cornea. Thus, the observation operation can be efficiently made by detecting a predetermined amount of movement of the observation container and turning on or enhancing the second light source. When the second light source is turned off during observation of the endothelium, the second light source may be turned on during observation of the epithelium, and when the second light source is turned on with a low light amount during observation of the endothelium, the second light source may be enhanced during observation of the epithelium.

Preferably, the light source control means of the light source control of the present invention turns off or dims the second light source during observation by the second imaging unit.

When the second imaging unit takes the entire image, light of the second light source may make the image invisible by the direct light. Thus, when the second imaging unit is selected, the second light source is turned off or dimmed. This can achieve a high-quality entire image.

According to the present invention, preferably, a temperature detecting unit configured to detect the temperature of the cornea for transplantation is provided, and the light source control means turns on or enhances the second light source when the temperature of the cornea for transplantation is lower than a predetermined value.

When the temperature of the cornea for transplantation is low, it may be difficult to acquire the cornea cell image. This is due to that irregularities occur on the surface of the cornea, and the first imaging unit cannot catch reflection of the slit light beam by specular reflection. Thus, when the temperature is lower than a predetermined value (for example, 20° C.), the second light source is turned on or enhanced. This can illuminate the cornea for transplantation from behind with scattered light, making the cornea cell image clearly visible.

### BRIEF DESCRIPTION OF THE DRAWINGS

FIG. 1 is a schematic view illustrating an example of configuration of an apparatus for observing cornea for transplantation in accordance with First embodiment;

FIG. 2 is a view illustrating configuration of an observation-container holding unit used in the apparatus for observing cornea for transplantation in accordance with First embodiment;

FIG. 3 is a block diagram illustrating control functions in the observation apparatus;

FIG. 4 is a block diagram illustrating functions of the control unit and components of the observation apparatus;

FIG. 5A are views illustrating switching between taking of a magnified image and taking of an entire image, and examples of display on a monitor screen;

FIG. 5B are views illustrating switching between taking of a magnified image and taking of an entire image, and examples of display on the monitor screen;

FIG. 5C is a view illustrating switching between taking of a magnified image and taking of an entire image, and an example of display on the monitor screen;

FIG. 6 is a schematic view illustrating an example of configuration of an apparatus for observing cornea for transplantation in accordance with Second embodiment;

FIG. 7 is a schematic view illustrating an example of configuration of an apparatus for observing cornea for transplantation in accordance with Third embodiment;

FIG. 8 is a schematic view illustrating an example of configuration of an apparatus for observing cornea for transplantation in accordance with Fourth embodiment;

FIG. 9 is a schematic view illustrating an example of configuration of an apparatus for observing cornea for transplantation in accordance with Fifth embodiment;

FIG. 10 is a schematic view illustrating an example of configuration of an apparatus for observing cornea for transplantation in accordance with Sixth embodiment; and

FIG. 11 is a schematic view illustrating an example of configuration of an apparatus for observing cornea for transplantation in accordance with Seventh embodiment.

### DETAILED DESCRIPTION OF THE PREFERRED EMBODIMENTS

An apparatus for observing cornea for transplantation and an observation system in accordance with embodiments of the present invention will be described below with respect to figures. The figures are schematic, and do not necessarily illustrate actual dimension ratio of components.

### Configuration of Apparatus for Observing Cornea for Transplantation in First Embodiment

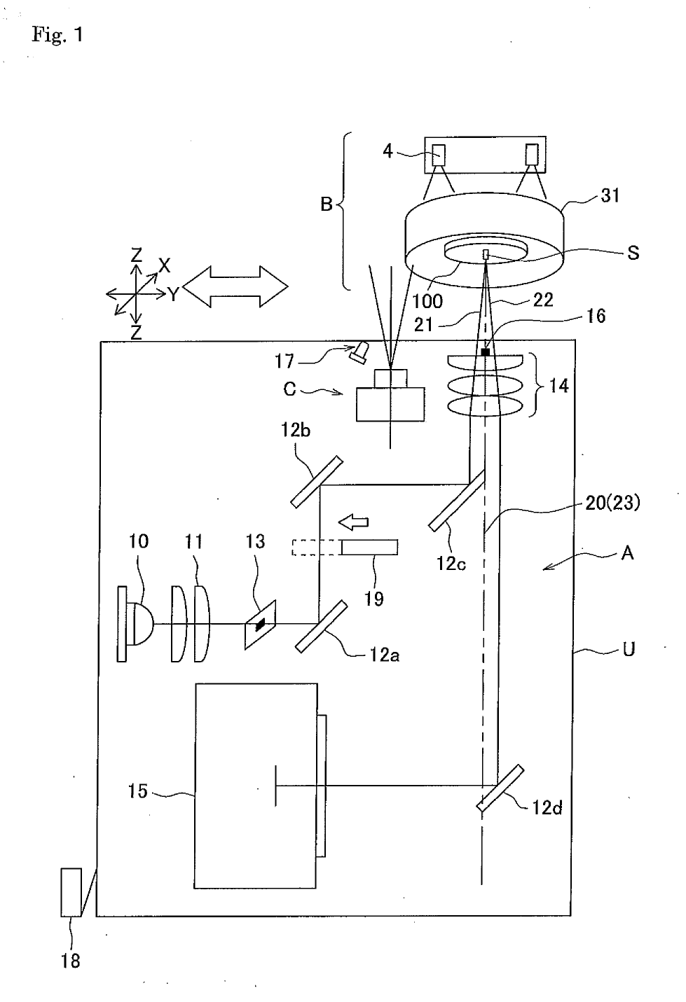

FIG. 1 is a schematic view illustrating an example of configuration of an apparatus for observing cornea for transplantation in accordance with First embodiment. The observation apparatus mainly includes a specular optical system A, an observation-container holding unit B, and a finder camera C (corresponding to a second imaging unit). In FIG. 1, X denotes a forward and rearward direction (direction perpendicular to sheet), Y denotes a right and left direction, and Z denotes a vertical direction. The same applies to other embodiments. The finder camera C and the specular optical system A constitute a unit U that can integrally move to right and left.

First, configuration of the specular optical system A will be described. The specular optical system A includes an illuminating optical system and an imaging optical system. The illuminating optical system includes a light source 10, condenser lens 11, mirrors 12a, 12b, and 12c, a slit 13, and an objective lens system 14. The imaging optical system includes the objective lens system 14, a mirror 12d, and a cornea observation camera 15 (corresponding to a first imaging unit). The cornea observation camera 15 is, for example, a CMOS camera or a CCD camera, which can observe a magnified image of a cornea for transplantation on a monitor (described later). In the observation apparatus illustrated in FIG. 1, the illuminating optical system and the imaging optical system commonly use the objective lens system 14, achieving miniaturization of the specular optical system A. Arrangement and the number of the mirrors and the number of condenser lenses may be changed as appropriate.

The light source 10 is, for example, a halogen lamp or an LED. A light beam from the light source 10 passes through the slit 13 and then, is incident on a predetermined observed point of a cornea for transplantation 100 via the objective lens system 14. The slit light beam passes through the objective lens system 14 with a deviation from an optical axis 20 of the objective lens system 14 in one direction (in FIG. 1, passes through a left half of the objective lens system 14), and is incident on the cornea for transplantation 100 with a small incident angle obliquely with respect to the optical axis 20 of the objective lens system 14, and a light flux having an effective illuminating optical axis 21. The slit light beam is secularly-reflected symmetrically with respect to the optical axis 20 of the objective lens system on the cornea for transplantation 100. The secularly reflected light beam (reflected image of the cornea for transplantation 100) is incident on the objective lens system 14, and passes the position opposite to the slit light beam with respect to the optical axis 20 of the objective lens system in the objective lens system 14 (in FIG. 1, a right half of the objective lens system 14). That is, the light beam is reflected on the mirror 12d as a light flux having an effective imaging optical axis 22 and then, forms an image on the cornea observation camera 15. A shield plate 16 provided at an upper end of the objective lens system 14 prevents incident light from interfering with reflected light.

FIG. 1 schematically illustrates the observation-container holding unit B in a three-dimensional manner when viewed from obliquely below. A slit light beam S is projected on the center of the cornea for transplantation 100, and L denotes a longitudinal direction of the slit light beam S. L is parallel to the X direction.

An intersection of the effective illuminating optical axis 21 and the effective imaging optical axis 22 constitutes an observed point, and the specular optical system A is configured such that the slit light beam S forms an image at the observed point. Therefore, it is important to position the cornea for transplantation 100 as an observed target at the observed point.

In this description, an axis that bisects an intersection angle of the effective illuminating optical axis 21 and the effective imaging optical axis 22 is defined as “observation basic axis 23”. In this embodiment, the objective lens system 14 is disposed such that the optical axis 20 matches the observation basic axis 23. Preferably, both the axes completely match each other. However, minutely unmatched axes fall within the scope of the present invention.

The finder camera C is, for example, a CMOS camera or a CCD camera that can take an entire image including the cornea for transplantation 100.

In First embodiment, there is provided a switching mechanism that switches between a state where the specular optical system A (objective lens system 14) is located immediately below an observation container 31 in the vertical direction, and a state where the finder camera C is located immediately below the observation container 31 in the vertical direction. A mechanism that moves the unit U configured of the finder camera C and the specular optical system. A in the right and left direction (Y direction) is adopted as the switching mechanism. Examples of the switching mechanism include an actuator such as a motor, and a power transmitting mechanism such as a gear. The switching mechanism may be automatic or manual.

There is provided a switching detection switch 18 that detects which of the specular optical system A or the finder camera Cis located below the observation container 31. In the example illustrated in FIG. 1, the switching detection switch 18 turns ON when the specular optical system A is located below the observation container 31, and turns OFF when the finder camera C is located below the observation container 31. This can be reversed. The switching detection switch 18 may be a mechanical switch, or a switch with an optical or magnetic detection mechanism. The switching detection switch 18 may be disposed at any suitable place.

An illuminating member 17 is provided near the finder camera C, and is used when the finder camera C takes an image. The illuminating member 17 serves to illuminate a region including the cornea for transplantation 100, and is an LED or a fluorescent lamp, for example. Under low light conditions, the illuminating member 17 is preferably used. The illuminating member 17 may be turned on and off by the operator, or may be automatically controlled by detecting the brightness of surroundings.

In connection with the detection of the switching detection switch 18, the illuminating member 17 may be automatically turned on when the finder camera C is used. The illuminating member 17 may be controlled with the detection of the brightness of surroundings according to the operator's preference.

An optical filter 19 can advance and retract in an optical path between the mirror 12a and the mirror 12b of the specular optical system A. For example, when the cornea is colored and analyzed, the optical filter 19 can advance into the optical path. During nonuse, the optical filter 19 retracts from the optical path.

### <Observation-Container Holding Unit>

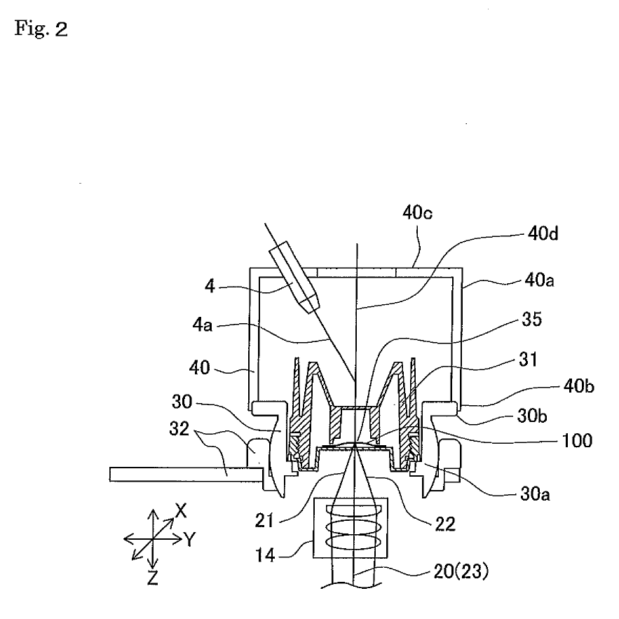

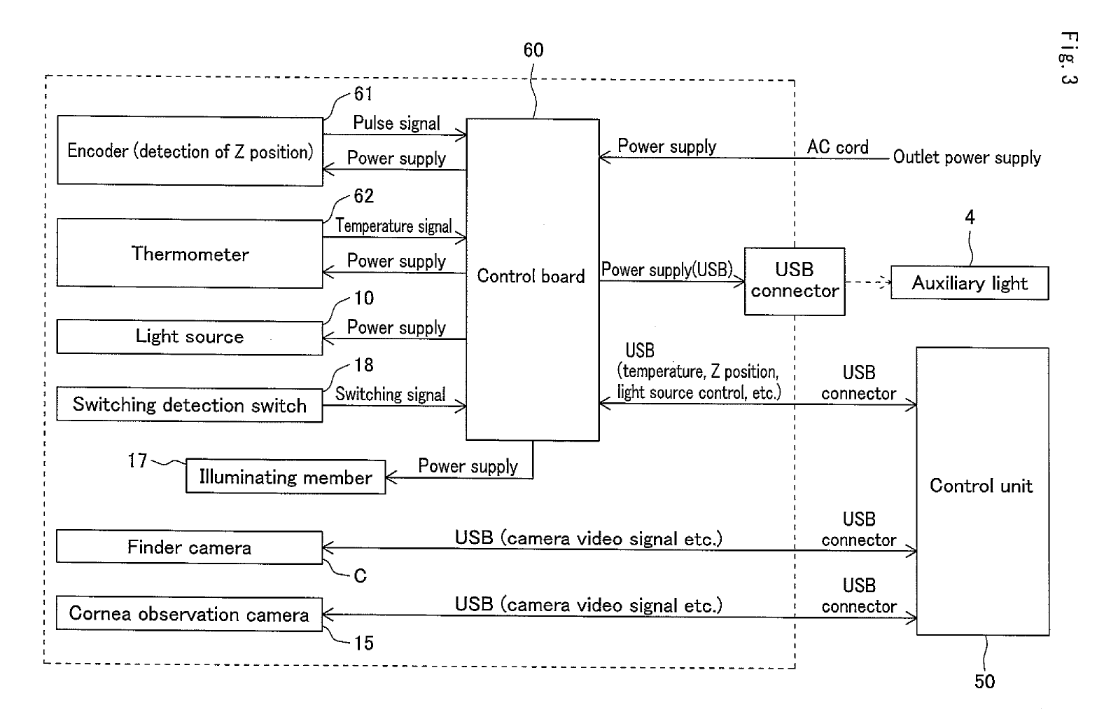

FIG. 2 illustrates the configuration of the observation-container holding unit B in FIG. 1 in more detail. A holding table 30 serves to hold the observation container 31 for containing the cornea for transplantation 100, the observation container 31 being placed at a predetermined place. The holding table 30 is opened so as not to obstruct the optical path of the specular optical system A. The observation container 31 serves to contain the cornea for transplantation 100, and stores the cornea for transplantation 100 in a predetermined liquid such as preservative medium. Generally, in the state illustrated in FIG. 2, the cornea for transplantation 100 is contained in the observation container 31 such that an epithelium faces upward, and an endothelium faces downward. Accordingly, the entire cornea is substantially an upwardly-facing spherical face. For example, the observation container 31 is substantially tubular, and can be placed on a mounting face 30a of the holding table 30.

A support table 32 can be moved forward and rearward, to right and left, and up and down (XYZ triple-axial directions) with respect to the observation basic axis 23 by an adjusting mechanism not illustrated (observation-container moving mechanism). This can move the observation container 31 in the triple-axial directions. The adjusting mechanism can set the observed position of the cornea for transplantation, and adjust the cornea for transplantation to the observed point (focusing point). Examples of the adjusting mechanism include a screw-type sliding mechanism. An encoder (not illustrated) for detecting movement in each of the triple-axial directions is provided. The adjusting mechanism may drive movement in the triple-axial directions manually or automatically by use of an actuator. Only movement in one of the triple-axial directions may be automatically actuated. For example, movement in the Z direction (vertically upward and downward) may be automatically actuated.

The support table 32 is a table having a female spherical face, and the holding table 30 is a table having a male spherical face. The holding table 30 and the support table 32 can be spherically fitted into each other, causing the holding table 30 to freely incline relative to the support table 32. Inclining the holding table 30 enables more suitable specular reflection on the cornea for transplantation 100. When the support table 32 moves in the triple-axial directions, with the holding table 30 being assembled to the support table 32, the holding table 30 and the support table 32 integrally move in the same direction.

The specific fitting mode between the holding table 30 and the support table 32 may be any mode as long as the holding table 30 can freely incline. In this manner, the observation container 31 is supported by the support table 32 via the holding table 30.

### <Auxiliary Light Source Unit>

The observation-container holding unit B includes an auxiliary light source unit placed on the holding table 30. The auxiliary light source unit includes a housing 40, and the housing 40 has a tubular body 40a, an opening 40b formed in one end of the tubular body 40a, and a top plate 40c provided at the other end of the tubular body 40a. The opening 40b of the housing 40 engages with an engaging face 30b of the holding table 30. Thus, the housing 40 can be rotatably attached to the holding table 30. The rotary axis is an axis of the tubular body 40a.

An oblong tubular auxiliary light source 4, an axis of which is inclined, is attached to the top plate 40c. The shape of the auxiliary light source 4 is not limited to the above-mentioned shape.

The housing 40 is shaped like a cap as a whole, contains the observation container 31, and is mounted on the holding table 30. The housing 40 is made of a material having a low translucency, eliminating the effect of disturbance light such as interior illumination and solar radiation. The specific shape of the housing 40 is not limited to the shape illustrated in FIG. 2.

With such configuration, the housing 40 is rotated with respect to the holding table 30, thereby rotating the auxiliary light source 4 integrally. The housing 40 is merely placed on the holding table 30 and thus, can be readily detached.

A tubular axis 40d of the housing 40 is arranged to match the observation basic axis 23. Preferably, the axes match completely and however, may slightly mismatch. When the holding table 30 is inclined, the tubular axis 40d may be inclined.

The auxiliary light source 4 has an auxiliary illuminating optical axis 4a inclined with respect to the observation basic axis 23, and illuminates the cornea for transplantation 100 from behind the specular optical system A. As described above, the orientation of inclination of the auxiliary illuminating optical axis 4a with respect to the observation basic axis 23 can be continuously changed by rotating the housing 40 about the tubular axis 40d.

The specific configuration of the auxiliary light source 4 and the auxiliary light source unit is not limited to the configuration illustrated in FIG. 2. For example, various embodiments as disclosed in Japanese Unexamined Patent Publication No. 2015-35999 can be adopted.

### <Control Function>

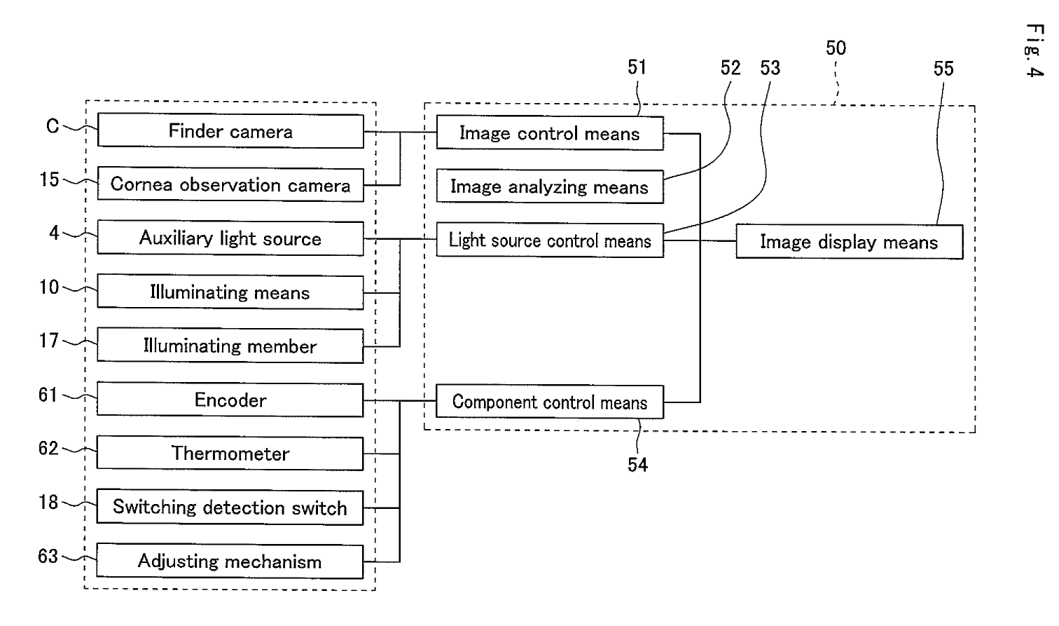

FIG. 3 is a block diagram illustrating mainly control functions in the observation apparatus. The observation apparatus includes a control board 60 (controller) for controlling each part. The control board 60 receives power from a commercial power supply.

The control board 60 receives a pulse signal from an encoder 61. The control board 60 feeds power to the encoder 61. The encoder 61 functions to detect movement of the observation container 31 (observation-container holding unit B) in the vertical direction (Z direction). A thermometer 62 (corresponding to a temperature detecting unit) is a sensor for measuring the temperature of the cornea for transplantation 100. The observation container 31 containing the cornea for transplantation 100 is set to an area in the observation apparatus, to measure the temperature of the cornea for transplantation 100. The thermometer 62 may be provided near the objective lens so as to face an observed portion unit of the observation container 31 on the holding table. This enables temperature measurement in an observation preparation state. The thermometer 62 sends a temperature signal to the control board 60. The thermometer 62 receives power from the control board 60. The light source 10 of the specular optical system A receives power from the control board 60. The switching detection switch 18 sends a switching signal to the control board 60. The auxiliary light source 4 receives power from the control board 60 via a USB connector. The illuminating member 17 receives power from the control board 60.

For example, a control unit 50 includes a personal computer and a dedicated software. The control unit 50 controls each component of the observation apparatus. The control board 60 is connected to the control unit 50 via a USB connector. The control board 60 sends various signal data (for example, temperature data, Z-position signal from the encoder) to the control unit 50. The control unit 50 sends various control signals (for example, turn-on, turn-off, dimming, enhancement of the light source 10 and the auxiliary light source 4) to the control board 60.

The finder camera C and the cornea observation camera 15 each are connected to the control unit 50 via a USB connector. Camera video signals from the imaging units are sent to the control unit 50 via the respective USB connectors. The control unit 50 transmits a control signal to each of the imaging units (the finder camera C and a cornea observation camera 15) via a USB connector.

FIG. 4 is a block diagram illustrating relation between control functions of the control unit 50 and the components of the observation apparatus. An image display means 55 is a monitor, typically, a liquid crystal monitor. An image control means 51 allows the monitor to display images taken by the cornea observation camera 15 and the finder camera C, and controls size and position of the displayed images. An entire image taken by the finder camera C and a magnified image of the cornea for transplantation taken by the cornea observation camera 15 are stored in a memory such as a hard disc as necessary. An image analyzing means 52 analyses the state of the cornea for transplantation cell and its scratch on the basis of the acquired image.

Alight source control means 53 controls the light source 10 and the auxiliary light source 4. The light source 10 is turned on when the specular optical system A is located below the observation container 31. Since it is no need to turn on the light source 10 when the finder camera C is located below the observation container 31, the light source 10 is turned off. Which the specular optical system A or the finder camera C is located below the observation container 31 can be determined based on the signal from the switching detection switch 18.

The auxiliary light source 4 is mainly used to observe the epithelium of the cornea for transplantation 100. To observe the endothelium, the light source 10 of the specular optical system A can eject a slit light beam to the cornea for transplantation 100, and take a magnified image of a designated portion. However, when observing the endothelium, the slit light beam of the specular optical system A may not be used. In this case, the cornea for transplantation 100 is illuminated from behind to acquire an image of the endothelium. Details of this are disclosed in Japanese Unexamined Patent Publication No. 2015-35999.

A general observation procedure of the cornea for transplantation 100 is as follows. First, the cornea observation camera focuses the position of the endothelium to observe the endothelium. The acquired magnified image (endothelial cell image) is recorded, and analyzed later by the image analyzing means 52 illustrated in FIG. 4. A focusing point of the endothelium becomes an origin in the vertical (Z) direction and thus, the Z direction of the encoder is reset to 0. Although the cornea observation camera focuses by moving the observation container 31 in the Z direction in this embodiment, the cornea observation camera also focuses by moving the entire specular optical system A in the Z direction.

Next, the observation container 31 (observation-container holding unit) is moved upward in the vertical direction. The adjusting mechanism (observation-container moving mechanism) moves the observation container 31. An average thickness of the cornea for transplantation 100 is previously known, and such thickness data is stored in a memory of the control unit 50. It is estimated that the epithelium is present near the position of the observation container 31 moved upward by the average thickness. An actual thickness of the cornea for transplantation can be measured by causing the cornea observation camera 15 to focus at the position of the epithelium. Thus, when the observation container 31 is moved upward after observation of the endothelium, the encoder 61 detects movement.

When a predetermined amount of movement is detected, the auxiliary light source 4 is turned on. An image is acquired using scattered light generated by turning on the auxiliary light source 4 to facilitate focusing on the epithelium. The turn-on timing may be set as appropriate based on 50%, 75%, 100%, or the like of the average thickness of the cornea for transplantation 100. The thickness of the cornea for transplantation can be measured based on the Z position indicated by the encoder at focusing on the epithelium.

When observing the endothelium, the auxiliary light source 4 is turned off or dimmed. In the case where the auxiliary light source 4 is turned off during observation of the endothelium, the auxiliary light source 4 is turned on during observation of the epithelium. In the case where the auxiliary light source 4 is turned on with a low light amount during observation of the endothelium, the auxiliary light source 4 is enhanced during observation of the epithelium.

When the finder camera C takes an entire image, the auxiliary light source 4 is turned off or dimmed. When an illumination light beam from the auxiliary light source 4 directly enters into the finder camera C, the image becomes blurred. Thus, the auxiliary light source 4 is turned off or dimmed so as not to affect the image quality.

When the finder camera C takes an image, the illuminating member 17 is turned on. The illuminating member 17 is controlled to illuminate the entire lower face of the observation container 31. A clear image can be acquired by turning on the illuminating member 17 especially when the side of lower face of the observation container 31 is dark (or a room in which the observation apparatus is installed has a darkness that is lower than a predetermined brightness).

To observe the endothelium, the auxiliary light source 4 is not basically used. However, when the temperature of the cornea for transplantation is low, the auxiliary light source 4 is preferably used. When the temperature of the cornea for transplantation is low, irregularities may occur on the surface of the cornea. For this reason, according to the observation method based on specular reflection, the slit light beam is not ideally reflected on the surface of the cornea, such that the cornea observation camera 15 cannot take an image. Thus, a clear image can be acquired by turning on or enhancing the auxiliary light source 4 to illuminate the endothelium of the cornea from behind.

A component control means 54 receives a movement signal from the encoder 61 to allow the light source control means 53 to control turn-on, turn-off, dimming, or enhancement of the light source. The component control means 54 receives temperature data from the thermometer 62 to allow the light source control means 53 to control the light source. The component control means 54 receives a signal from the switching detection switch 18 to switch between an image signal from the finder camera C and an image signal from the cornea observation camera 15. One of the image signal is displayed on the monitor.

An adjusting mechanism 63 is an observation-container moving mechanism that adjusts the position of the observation container 31 (observation-container holding unit B) in the XYZ direction.

### <Switching Operation>

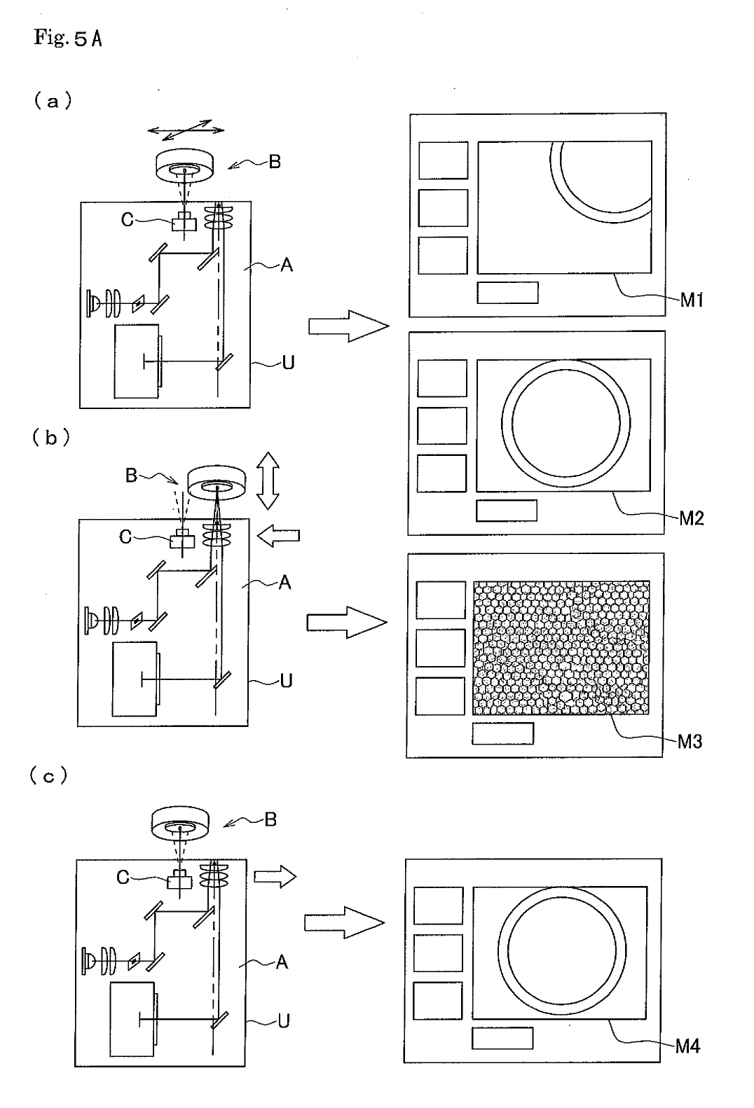

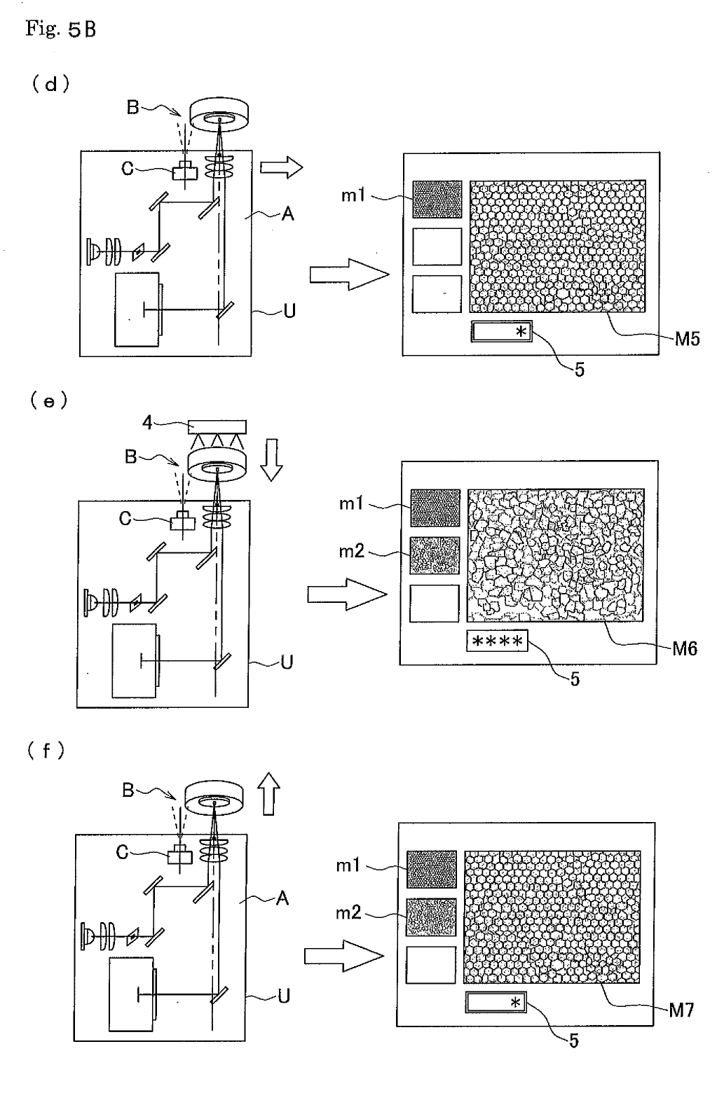

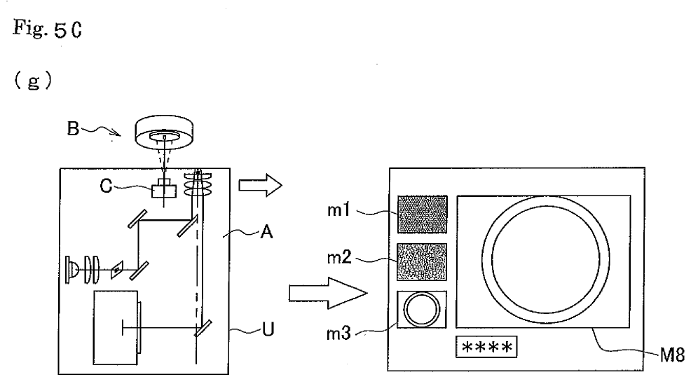

Next, a switching operation between taking of a magnified image and taking of an entire image will be described with reference to FIG. 5. The left side in FIG. 5 illustrates the state of the observation apparatus. The right side illustrates examples of display on a monitor screen.

First, the unit U is set to a state (a) in FIG. 5A. In this state, the finder camera C is located below the observation container 31 in the vertical direction.

First, the finder camera C views the observation container 31 (S1). The state is displayed as an entire image M1 on the monitor screen. A double circle displayed on the screen is the entire image of the cornea for transplantation. On the screen, the cornea for transplantation is not located at the center of the entire image M1. The position is adjusted in the XY direction while viewing the entire image on the monitor (S2).

For example, the center of the cornea for transplantation 100 is moved to the center (reference position) of the entire image (S3). The state where movement is completed is displayed as an entire image M2 on the monitor. The taken image can be stored in a memory by performing a predetermined operation. As described above, when taking the entire image, the finder camera C is located below the observation container 31 in the vertical direction. Thus, for example, when the observation container 31 is vertically moved, the position is advantageously not displaced. This also prevents deformation of the taken image.

Next, the camera is switched to the cornea observation camera (S4). (b) in FIG. 5A illustrates a state where the unit U is moved, and the specular optical system. A is located below the observation container 31 in the vertical direction. By moving the unit U and detecting switching by the switching detection switch 18, a switched image from the camera is displayed. When the Z position is not correct (no focusing), nothing is viewed. By moving the unit up or down in the Z direction, the position (focusing point) of the endothelium is searched (S5). Here, when the observed endothelium is dark, the holding table 30 is inclined for adjustment. A brightest and clearest image can be acquired when the surface of the endothelium is perpendicular to the optical axis 20. A magnified image M3 is displayed on the monitor.

After focusing at the endothelium (S6), the camera is switched to the finder camera C (S7). (c) in FIG. 5A illustrates this state. The unit U is moved, and the finder camera C is located below the observation container 31 again. At this time, an entire image M4 is displayed on the monitor. Using this image, the observed position is observed. This is due to that the observed position may be displaced with inclination of the holding table 30. In the case of displacement, the procedure returns to (S2).

When the observed position is correct, the unit U is moved, and the camera is switched to the cornea observation camera (S8). (d) in FIG. 5B illustrates this state. Here, it is checked whether a magnified image M5 of the endothelium is well-focused, and the image is stored. The stored image is displayed as a small image m1 in the left side on the monitor screed. The position in the Z direction by the encoder is reset (S9). The position in the Z direction is displayed in a display area 5 on the monitor screen. Upon reset, a numerical value becomes 0.

Next, to cause the cornea observation camera to properly focus on the epithelium of the cornea for transplantation 100, the observation container 31 is moved downward in the vertical direction (S10). (e) in FIG. 5B illustrates this state. With movement in the Z direction, the numerical value in the display area 5 also changes. When observing the epithelium, the auxiliary light source 4 is turned on to illuminate the cornea for transplantation 100 from behind. The auxiliary light source 4 is turned on (enhanced) during movement in the Z direction, facilitating observation. A taken epithelium image is displayed as a magnified image M6. When the camera focuses on the epithelium, the position in the Z direction is checked using the display area 5 (S11). The numerical value corresponds to the thickness of the cornea for transplantation 100. The magnified image of the epithelium in focus is stored. The stored image is displayed as m2. Cornea thickness data is also stored.

Next, the observation container 31 is moved again in the Z direction (vertically upward) to the position at which the camera focuses on the endothelium (S12). (f) in FIG. 5B illustrates this state. A magnified image M7 of the endothelium is displayed again on the monitor.

Next, the unit U is moved, and the camera is switched to the finder camera C. (g) in FIG. 5C illustrates this state. An entire image M8 is displayed on the monitor. The entire image M8 is stored. The stored image is displayed as m3. This image corresponds to location information on the taken cornea cell.

The center of the cornea for transplantation as well as surroundings of the cornea can be observed. Since the holding table 30 of the observation-container holding unit B spherically engages with the support table 32, the entire cornea for transplantation is held by the upward-facing spherical face. Thus, surroundings of the cornea can be observed while keeping the focused state by inclining the holding table 30.

When observing the epithelium, the auxiliary light source 4 is turned on (enhanced). However, also when the temperature of the cornea for transplantation is low (for example, 20° C. or lower), the auxiliary light source 4 is preferably turned on (enhanced).

The operator can determine a timing at which the image is stored as appropriate. The operator can also determine the number of stored images.

The relative position between the finder camera C and the cornea observation camera 15 is previously adjusted such that the center of the entire image matches the center of the magnified image.

### Second Embodiment

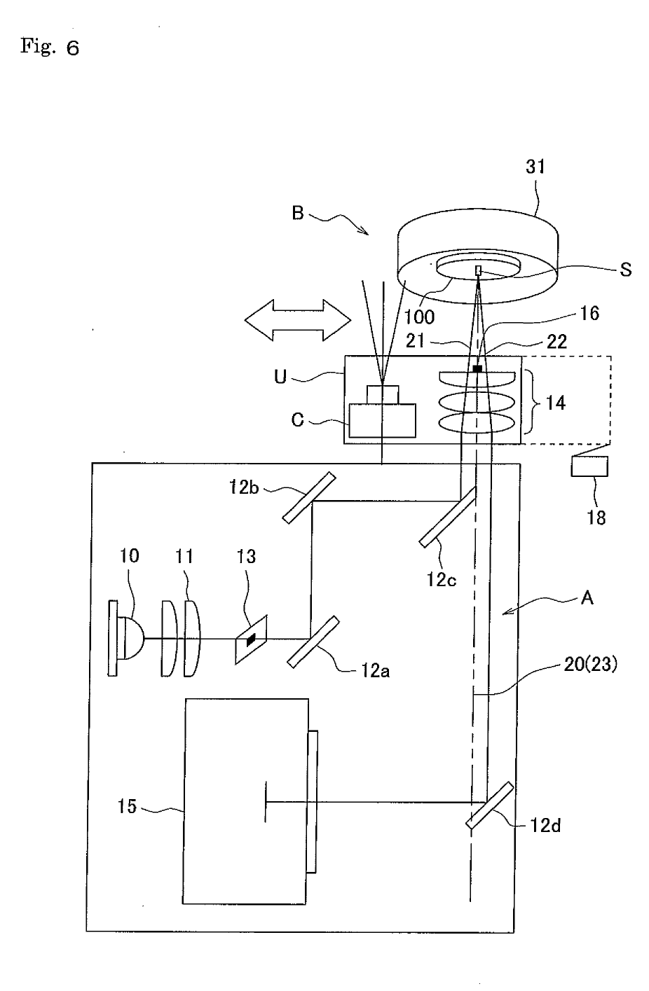

FIG. 6 illustrates an example of configuration of an apparatus for observing cornea for transplantation with respect to Second embodiment. With this configuration, the unit U is configured of the finder camera C and the objective lens system 14 constituting the specular optical system A (corresponding to a part of the specular optical system A). As in First embodiment, the switching detection switch 18 for detecting switching of the unit U is provided. The same components of the observation apparatus in this embodiment as those in First embodiment are given the same reference numerals, and description thereof is omitted. As compared to First embodiment, the unit U is smaller and thus, a moving load is smaller.

### Third Embodiment

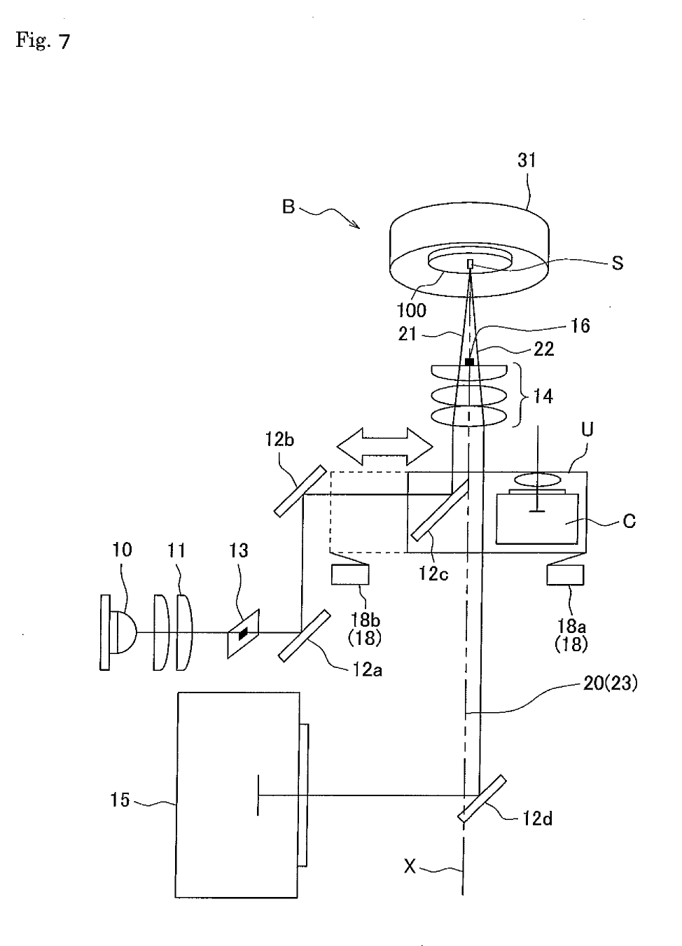

FIG. 7 illustrates an example of configuration of an apparatus for observing cornea for transplantation in accordance with Third embodiment. With this configuration, the unit U is configured of the finder camera C and the mirror 12c constituting the specular optical system A (corresponding to a part of the specular optical system A). The switching detection switch 18 for detecting switching of the unit U includes a first switch 18a and a second switch 18b. The two switches detect a terminal end of a moving range of the unit U. Other embodiments may include two switches. By moving the unit U from the state illustrated in FIG. 7 to the left, the finder camera C can be set below the observation container 31 in the vertical direction.

### Fourth Embodiment

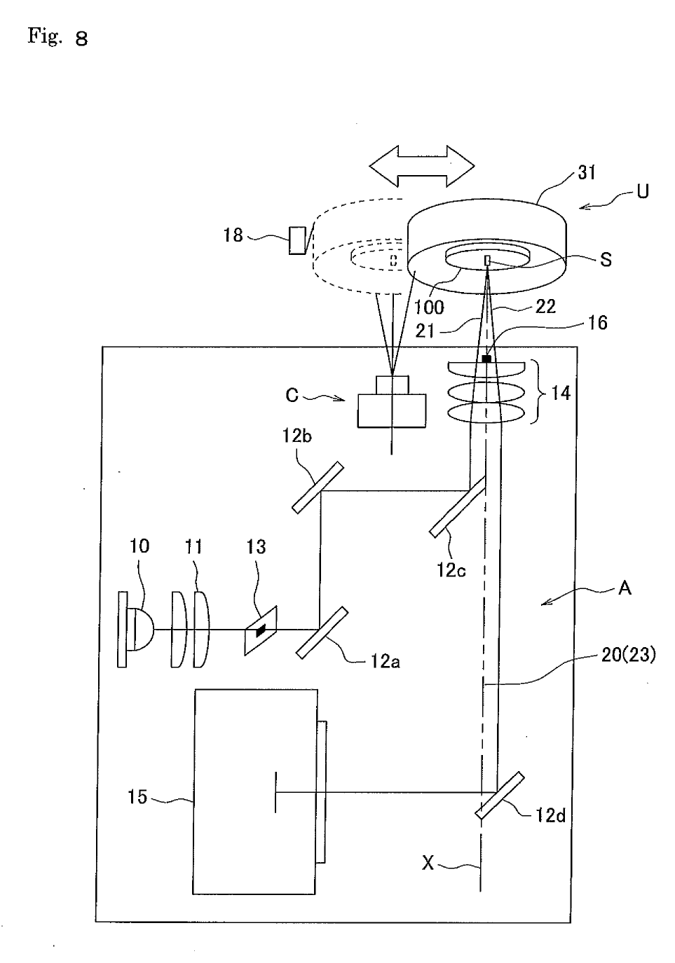

FIG. 8 illustrates an example of configuration of an apparatus for observing cornea for transplantation in accordance with Fourth embodiment. With this configuration, the unit U is the observation-container holding unit B including the observation container 31. The finder camera C can be set below the observation container 31 in the vertical direction by moving the unit U (observation-container holding unit B) from the state illustrated in FIG. 8 to the left. In this case, the observation-container moving mechanism functions as a switching mechanism. The container on the observation-container moving mechanism or the entire observation-container moving mechanism may be moved by a distance between optical axes of two cameras. Even when observation is switched, correlation of the observed positions can be kept.

### Fifth Embodiment

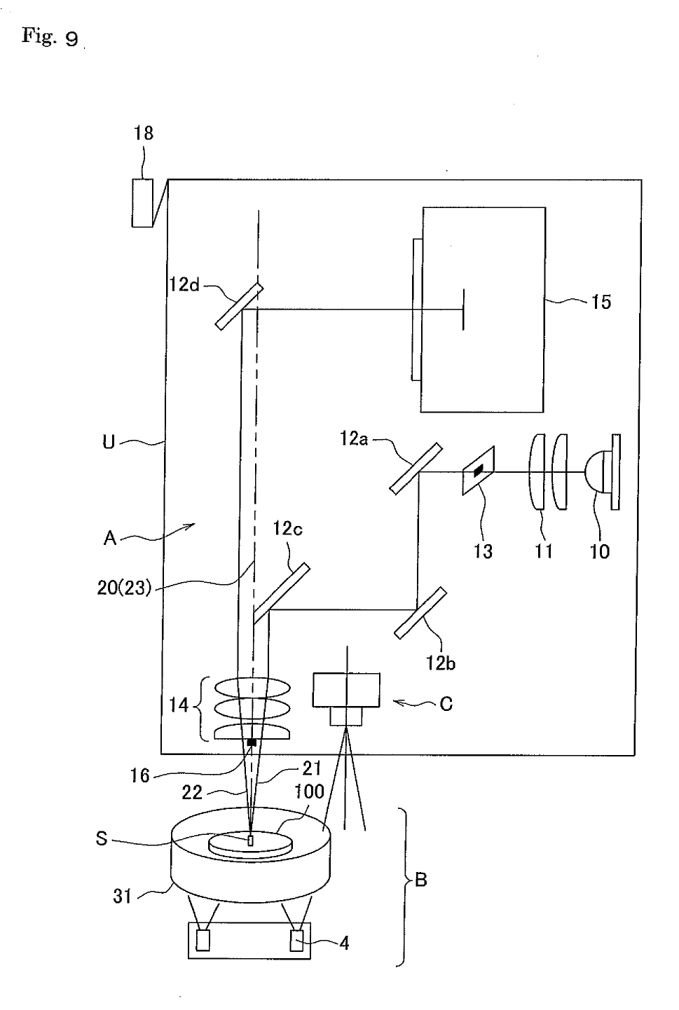

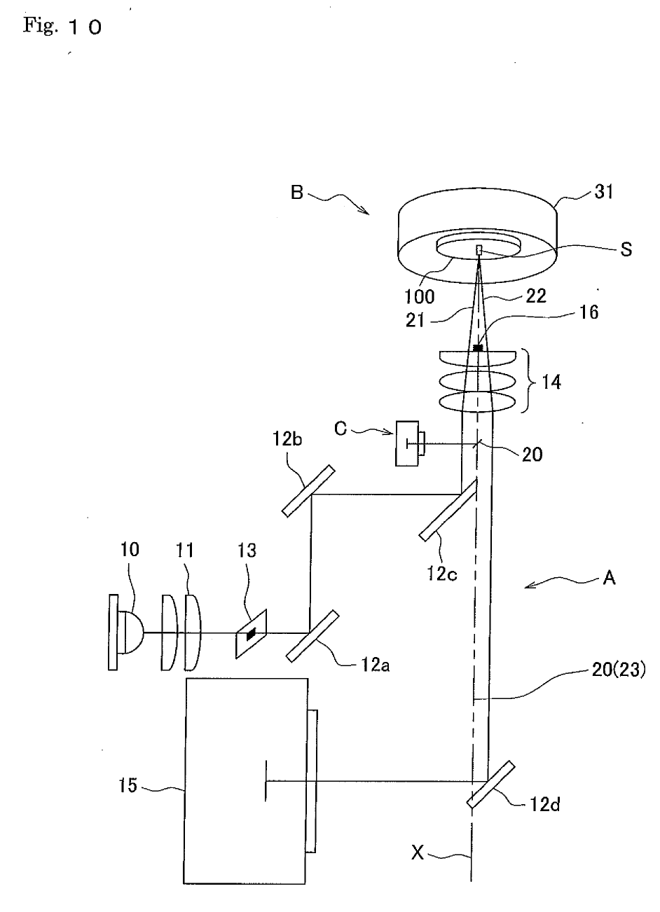

FIG. 9 illustrates an example of configuration of an apparatus for observing cornea for transplantation in accordance with Fifth embodiment. In this embodiment, as opposed to First embodiment illustrated in FIG. 1, the specular optical system A and the finder camera Care disposed in an upper side, and the observation-container holding unit B (observation container 31) is disposed in a lower side. Configuration and functions of the members are the same as those in First embodiment, except that the members are flipped vertically. In FIG. 9, the specular optical system A is set above the observation container 31 in the vertical direction. By moving the unit U to the left, the finder camera C can be set above the observation container 31 in the vertical direction. Also in the embodiments other than First embodiment, the vertically-flipped configuration can be adopted.

### Sixth Embodiment

FIG. 10 illustrates an example of configuration of an apparatus for observing cornea for transplantation in accordance with Sixth embodiment. As distinct from First to Fifth embodiments, this embodiment has no moving component. A small mirror 20 is provided below the objective lens system 14 of the specular optical system A, and the mirror reflects apart of a light beam, and guides the reflected beam to the finder camera C. With this configuration, the entire image can be acquired without interfering an optical path for observing the magnified image. With such configuration, no mechanical switching mechanism is present, achieving a switching mechanism without mechanical movement. For example, the magnified image and the entire image are switched on the monitor screen using a software. Such switching belongs to the switching mechanism of the present invention. In this case, for example, the finder camera C is provided with an infrared filter, the shield plate 16 uses a visible light-shielding and infrared-transmitting filter, and the illuminating member 17 is infrared light such that the shield plate 16 does not interfere with the entire image.

### Seventh Embodiment

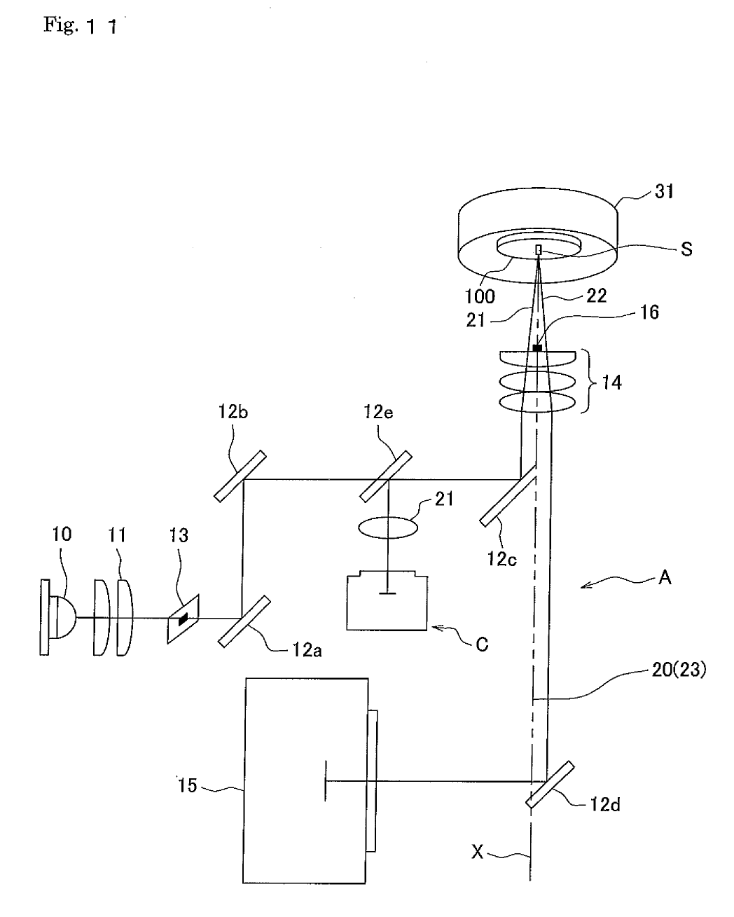

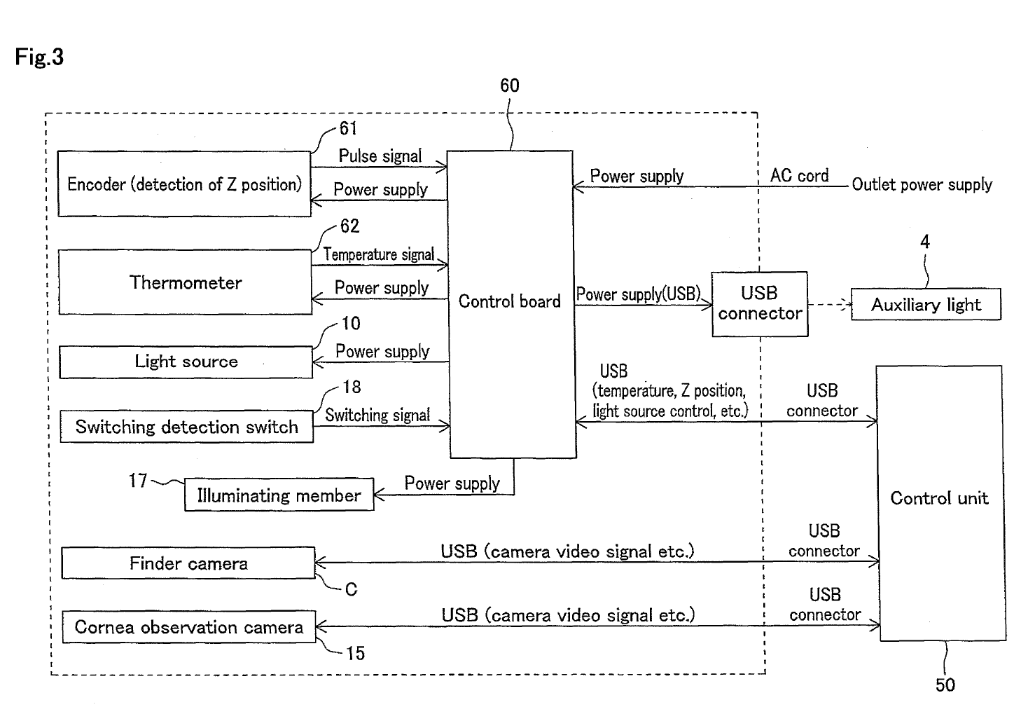

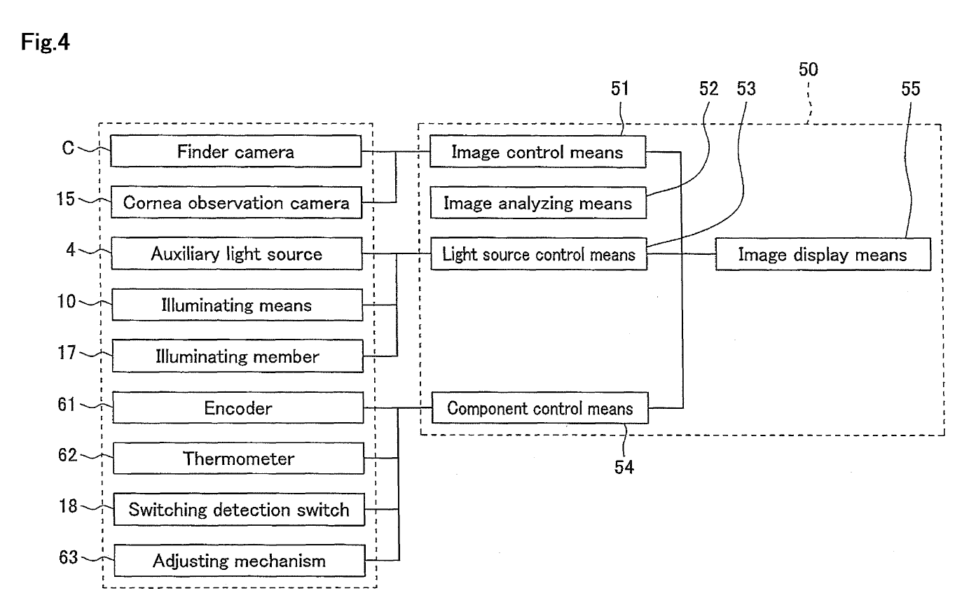

FIG. 11 illustrates an example of configuration of an apparatus for observing cornea for transplantation in accordance with Seventh embodiment. Like Sixth embodiment, this embodiment has no moving component. A half mirror 12e is provided between the mirror 12b and the mirror 12c, and a part of a light beam is taken into the finder camera C. A lens 21 is disposed on the side of the front surface of the finder camera C. A transmittance of the half mirror 12e can be appropriately set so as not to affect observation of the magnified image, for example, 50%. Also in this embodiment, the finder camera C is provided with an infrared filter, the shield plate 16 uses a visible light-shielding and infrared-transmitting filter, and the illuminating member 17 is infrared light such that the shield plate 16 does not interfere with the entire image.

### Other Embodiments

Display on the monitor screen in FIG. 5 is merely example, and the present invention is not limited to the example.

What is claimed is:

1. An apparatus for observing cornea for transplantation comprising:
a support table configured to support an observation container containing a cornea for transplantation as an observed target;
a specular optical system including an illuminating optical system and an imaging optical system, the specular optical system having a first light source and a first imaging unit, and being configured to acquire and observe a magnified image of the cornea for transplantation from above or below the observation container in the vertical direction by specular reflection;
a second imaging unit configured to an entire image from above or below the observation container in the vertical direction, the entire image including a region where the cornea for transplantation is contained; and
a switch configured to selectively switch between the first imaging unit and the second imaging unit to acquire the magnified image or the entire image.

2. The apparatus for observing cornea for transplantation according to claim 1, wherein
the switch has a mechanism configured to integrally move the second imaging unit and at least a part of the specular optical system.

3. The apparatus for observing cornea for transplantation according to claim 1, wherein
the switch has a mechanism configured to integrally move an objective lens system constituting the specular optical system and the second imaging unit.

4. The apparatus for observing cornea for transplantation according to claim 1, wherein
the switch has a mechanism configured to integrally move the entire specular optical system and the second imaging unit.

5. The apparatus for observing cornea for transplantation according to claim 1, wherein
the switch has a mechanism configured to advance and retract the second imaging unit in an optical path of the specular optical system.

6. The apparatus for observing cornea for transplantation according to claim 1, wherein
the switch has a mechanism configured to move the observation container.

7. The apparatus for observing cornea for transplantation according to claim 1, further comprising a switching detector configured to detect switching of the imaging unit by the switch.

8. The apparatus for observing cornea for transplantation according to claim 1, further comprising:
a second light source configured to illuminate the cornea for transplantation from above or below the observation container; and
an observation-container moving mechanism configured to move the observation container in a thickness direction of the cornea for transplantation, wherein
an endothelium, an epithelium, a portion between the endothelium and the epithelium of the cornea for transplantation can be observed.

9. A system for observing cornea for transplantation, comprising:
the apparatus for observing cornea for transplantation according to claim 1; and
a control unit configured to control operation of each component of the observation apparatus, wherein
the control unit has a light source control configured to turn on or enhance the second light source when a predetermined amount of movement of the observation container by the observation-container moving mechanism to observe an epithelium is detected.

10. The system for observing cornea for transplantation according to claim 9, wherein
the light source control turns off or dims the second light source during observation by the second imaging unit.

11. The system for observing cornea for transplantation according to claim 9, further comprising a temperature detecting unit configured to detect the temperature of the cornea for transplantation, wherein
the light source control turns on or enhances the second light source when the temperature of the cornea for transplantation is lower than a predetermined value.

12. The system for observing cornea for transplantation according to claim 10, further comprising a temperature detecting unit configured to detect the temperature of the cornea for transplantation, wherein
the light source control turns on or enhances the second light source when the temperature of the cornea for transplantation is lower than a predetermined value.

13. A method for observing a cornea for transplantation comprising:
supporting an observation container containing a cornea for transplantation as an observed target on a support table;
acquiring and observing a magnified image of the cornea for transplantation from above or below the observation container in the vertical direction by specular reflection;
imaging an entire image from above or below the observation container in the vertical direction, the entire image including a region where the cornea for transplantation is contained; and
selectively switching between acquiring the magnified image or the entire image.

14. The method for observing cornea for transplantation according to claim 13, wherein the selective switching comprises integrally moving an imaging unit configured to image the entire image and at least apart of a specular optical system configured to image the magnified image.

15. The method for observing cornea for transplantation according to claim 14, wherein the selective switching comprises integrally moving an objective lens system of the specular optical system and the imaging unit configured to image the entire image.

16. The method for observing cornea for transplantation according to claim 14, wherein the selective switching comprises integrally moving the imaging unit configured to image the entire image and the entire specular optical system.

17. The method for observing cornea for transplantation according to claim 14, wherein the selective switching comprises advancing and retracting the imaging unit configured to image the entire image in an optical path of the specular optical system.

18. The method for observing cornea for transplantation according to claim 13, wherein the selective switching comprises moving the observation container.

19. The method for observing cornea for transplantation according to claim 14, further comprising detecting switching of the imaging units.

20. The method for observing cornea for transplantation according to claim 13, further comprising:
illuminating the cornea for transplantation from above or below the observation container;
moving the observation container in a thickness direction of the cornea for transplantation; and
observing an endothelium, an epithelium, a portion between the endothelium and the epithelium of the cornea for transplantation.
